(19) United States
(12) Patent Application Publication
(10) Pub. No.: US 2014/0336577 A1
(43) Pub. Date: Nov. 13, 2014

(54) AUTO-INJECTOR SYRINGE

(71) Applicant: Jewis J. McCoy, Kansas City, MO (US)

(72) Inventor: Jewis J. McCoy, Kansas City, MO (US)

(21) Appl. No.: 14/272,012

(22) Filed: May 7, 2014

Related U.S. Application Data
(60) Provisional application No. 61/820,490, filed on May 7, 2013.

(51) Int. Cl. A61M 5/50

(52) U.S. Cl. CPC A61M 5/5086; A61M 2205/3303
USPC 604/111

(57) ABSTRACT

An automatic injector syringe with a red detector for automatically deploying medicine into a patient's body. The syringe is intended for use by non-medical profession personnel or the patients themselves. A red sensor instructs a microprocessor to inject the medication and retract the syringe upon indication of blood within the syringe being detected by the red sensor.

### CROSS-REFERENCE TO RELATED APPLICATION

This application claims priority in U.S. Provisional Patent Application Ser. No. 61/820,490, filed May 7, 2013, which is incorporated herein by reference.

### BACKGROUND OF THE INVENTION

1. Field of the Invention

The present invention relates generally to an auto-injector syringe apparatus, and more specifically to an auto-injector syringe intended to increase ease of use and reduce pain and discomfort when using the auto-injector syringe.

2. Description of the Related Art

Auto-injector syringes are intended for self-use by patients or for use by untrained medical personnel. The intent of the auto injector is to remove errors when injecting medicine into the body.

Most auto-injectors are spring-loaded syringes. These have a single pressure level, the spring can wear out, and the auto-injector is always capable of accidentally discharging, which can be costly and dangerous.

What is needed is an auto-injector syringe capable of simplifying the auto-injection process and improving usefulness of auto-injector syringes over the prior art.

Heretofore there has not been available an auto-injector syringe with the advantages and features of the present invention.

### BRIEF SUMMARY OF THE INVENTION

The present invention generally provides an auto-injector syringe intended for self-use injection of medication into the body in various locations, such as the buttocks, arms, and legs. The depth of injection may also vary. The present invention is powered by a motor which will not discharge unless the presence of blood is sensed prior to injection.

### BRIEF DESCRIPTION OF THE DRAWINGS

FIG. 1 is a sectional view of an embodiment of the present invention.

FIG. 2 is a detailed sectional view of an embodiment of the present invention.

FIG. 3 is a side sectional view of portion of an embodiment of the present invention.

FIG. 4 is a side sectional view of portion of an embodiment of the present invention.

FIG. 5 is a top sectional plan view of a portion of an embodiment of the present invention.

FIG. 6 is a top sectional plan view of a portion of an embodiment of the present invention.

FIG. 7 is a top plan view of a portion of an embodiment of the present invention.

FIG. 8 is a side sectional view of a portion of an embodiment of the present invention.

FIG. 9 is a side sectional view of a portion of an embodiment of the present invention.

FIG. 10 is an alternative view of a portion of an embodiment of the present invention.

FIG. 11 is a side sectional view of a portion of an embodiment of the present invention.

FIG. 12 is a three-dimensional isometric view of a portion of an embodiment of the present invention.

FIG. 13 is a top sectional plan view of a portion of an embodiment of the present invention.

FIG. 14 is a side sectional view of a portion of an embodiment of the present invention.

FIG. 15 is a side sectional view of a portion of an embodiment of the present invention.

FIG. 16 is a top sectional plan view of a portion of an embodiment of the present invention.

FIG. 17 is a side sectional view of a portion of an embodiment of the present invention.

FIG. 18 is a side sectional view of a portion of an embodiment of the present invention.

FIG. 19 is a top sectional plan view of a portion of an embodiment of the present invention.

FIG. 20 is a top sectional plan view of a portion of an embodiment of the present invention.

FIG. 21 is a side sectional view of a portion of an embodiment of the present invention.

FIG. 22 is a top plan view thereof.

FIG. 23 is a bottom plan view thereof.

FIG. 24 is a side elevational view of a portion of an embodiment of the present invention.

FIG. 25 is a side sectional view of a portion of an embodiment of the present invention.

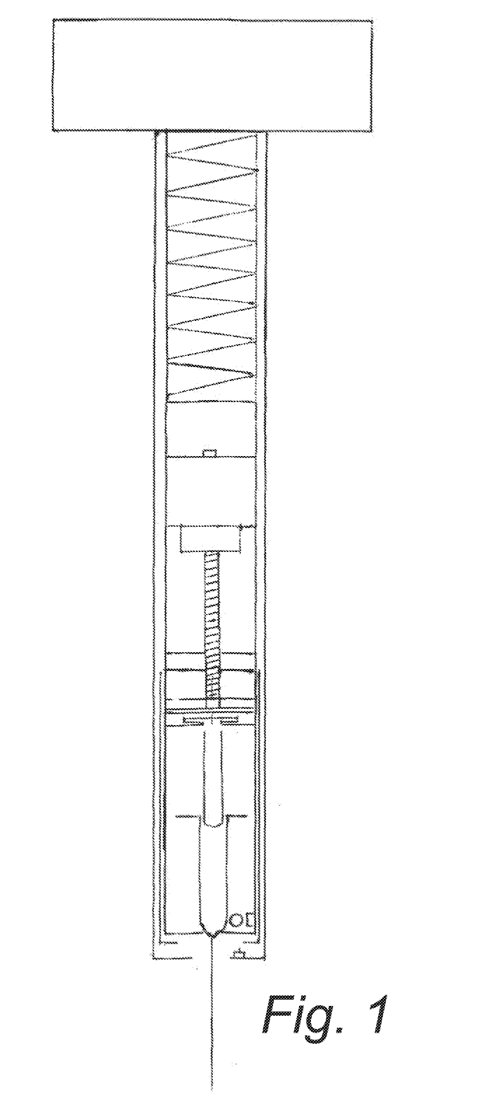

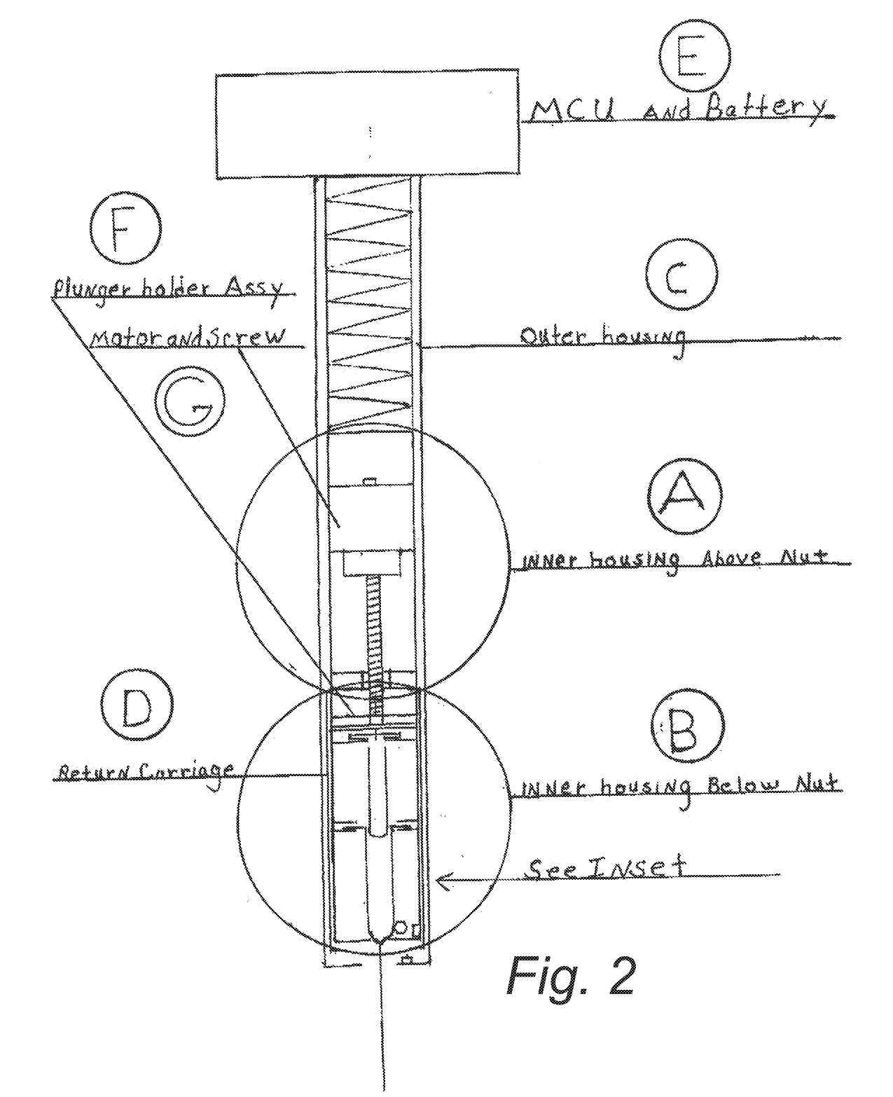

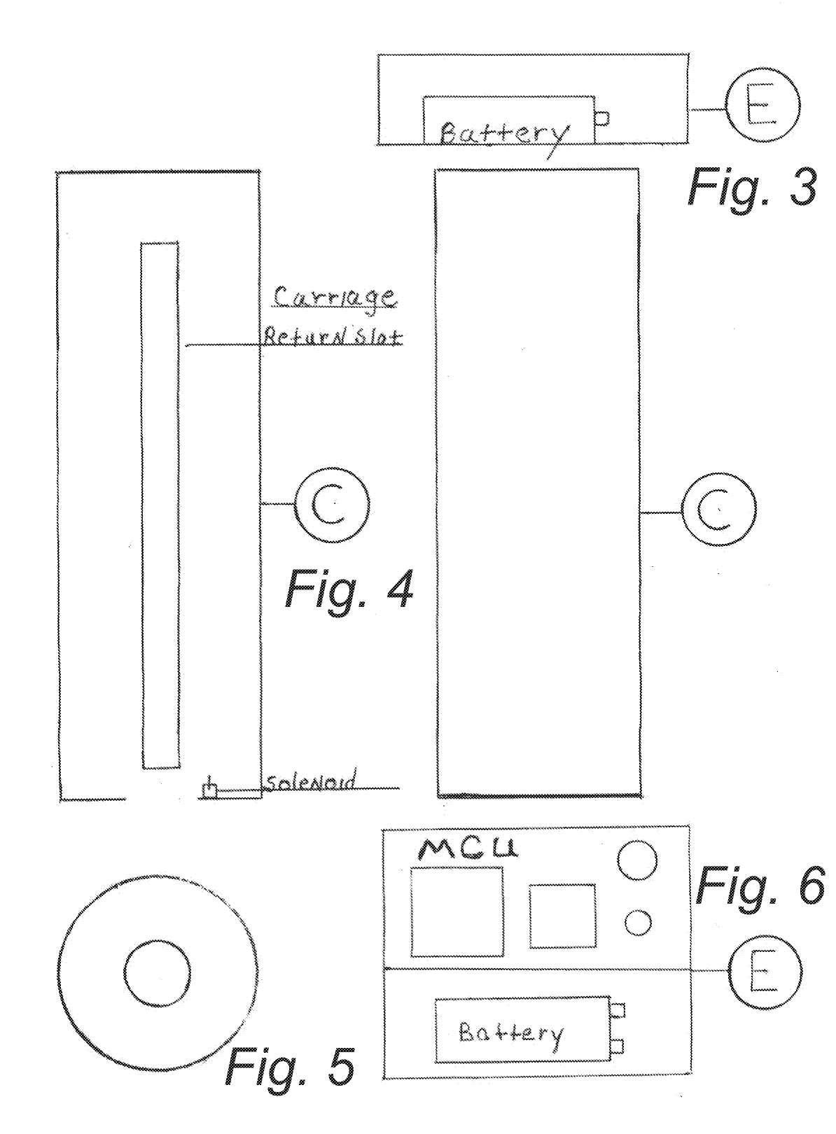

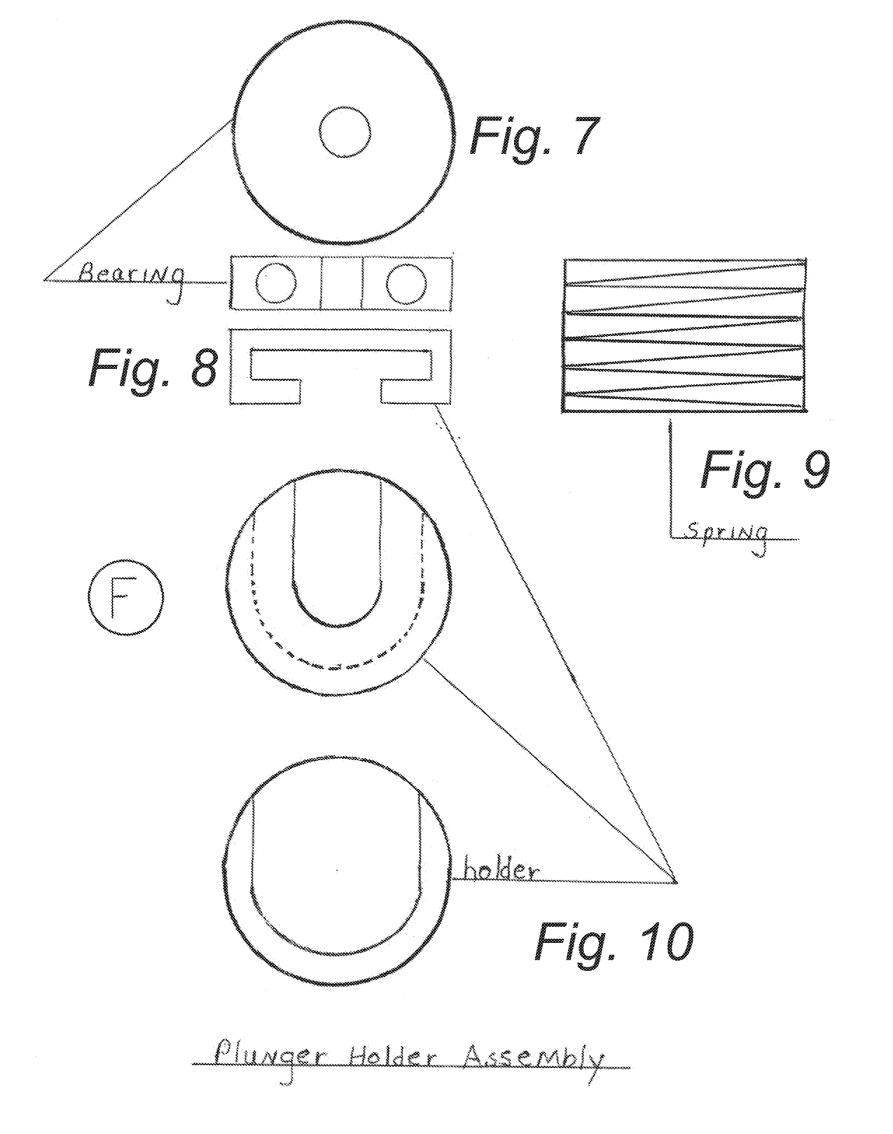

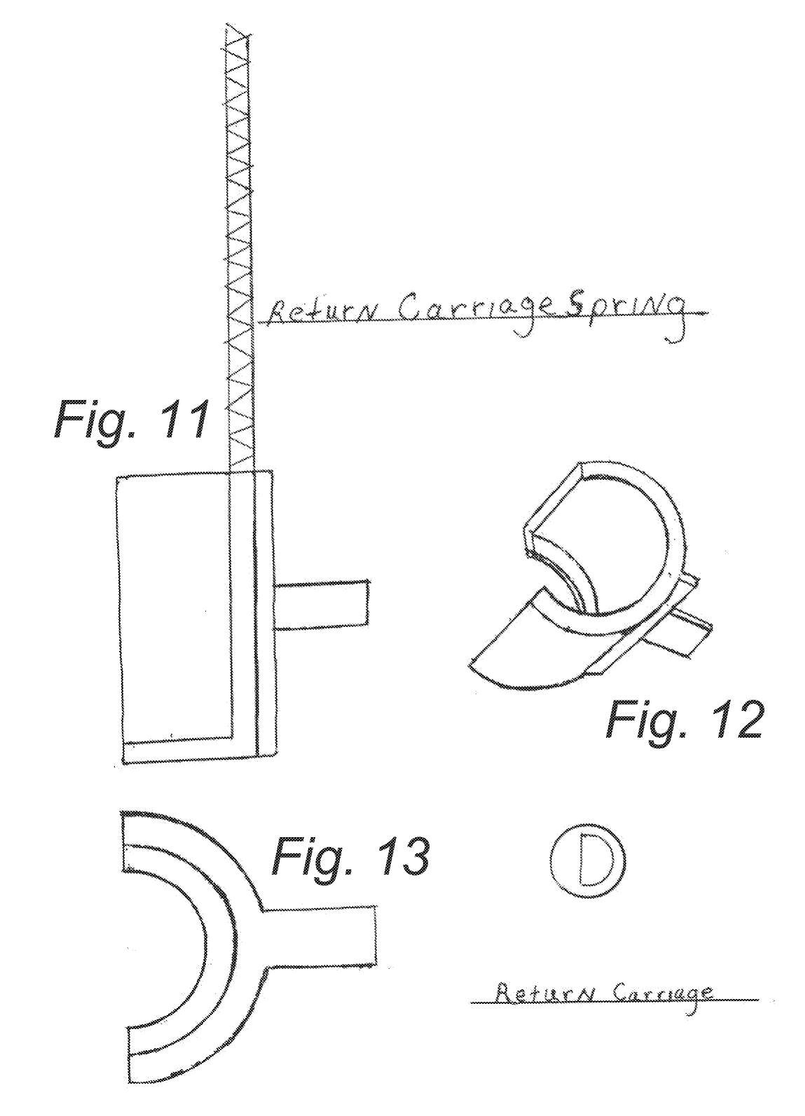

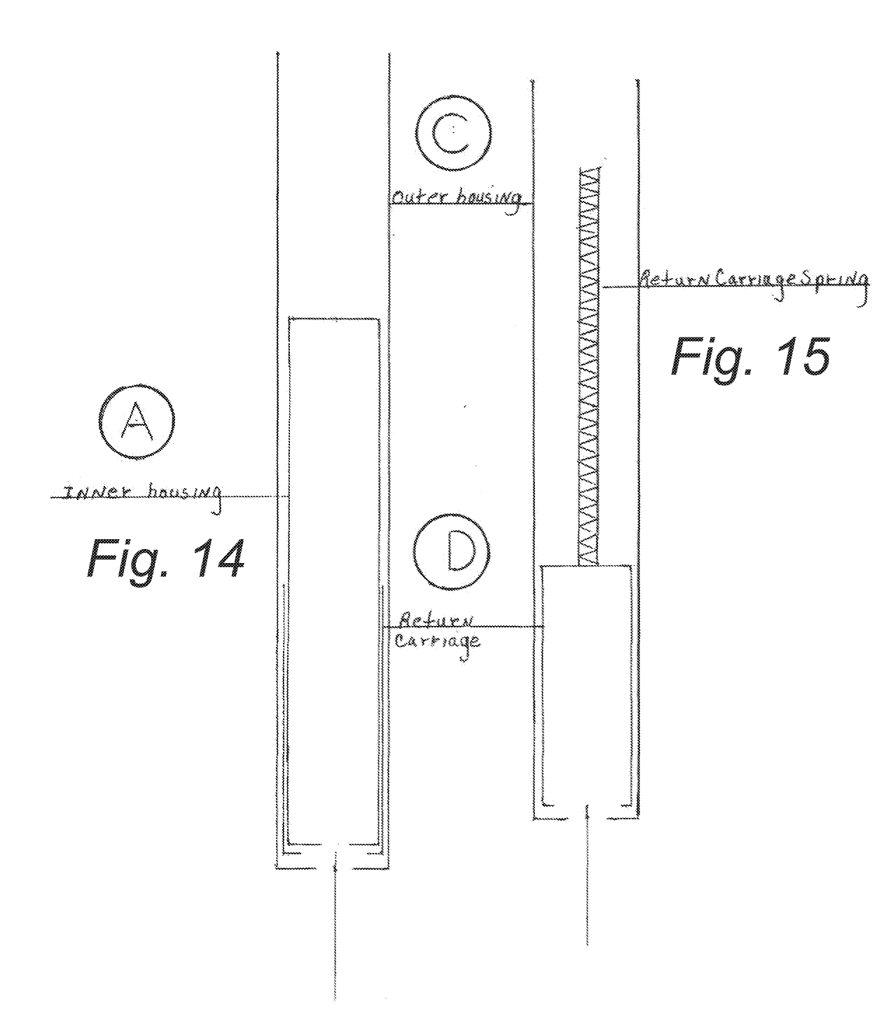

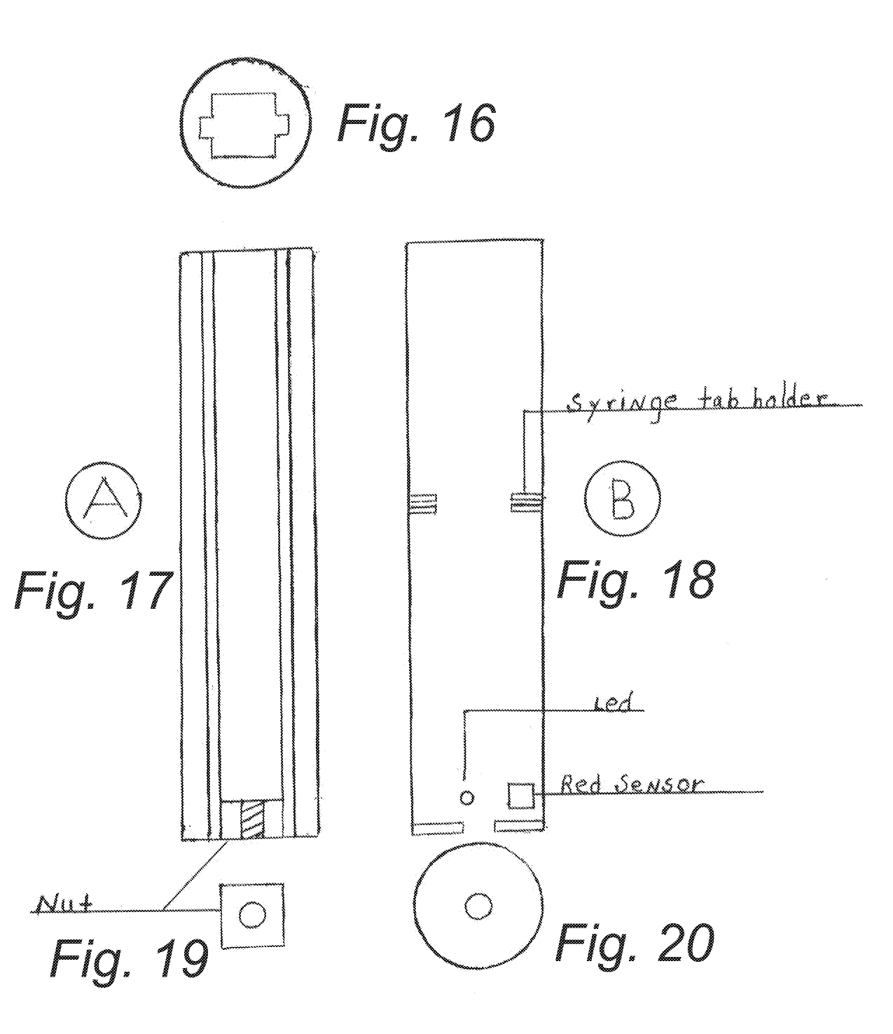

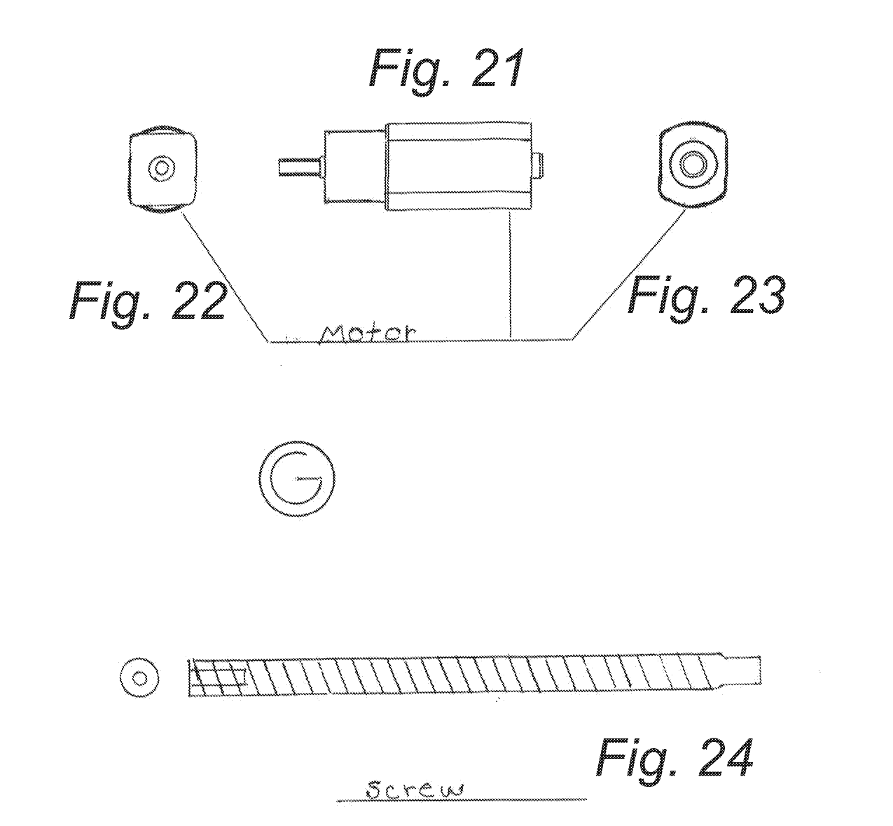

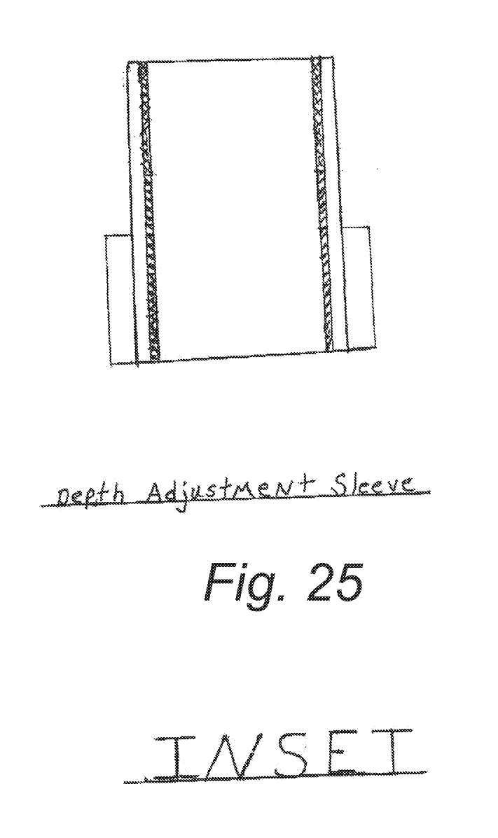

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment

The figures present an auto-injector syringe which is powered by a motor. A single push of a button will cause the needle to penetrate slightly such as to aspirate a blood vessel. A sensor will determine whether blood has been drawn into the syringe. If the sensor senses blood, the needle will descend, inject, and be retracted. Otherwise the needle will not inject. The red/blood sensor acts as a safety deterrent that previous auto-injectors lack.

The red sensor is connected to a microprocessor which further controls the motor function. The present invention can be set to inject at various depths for different medication applications.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. An automatic injector syringe comprising:
a housing configured to receive a syringe, said housing including a proximal end and a distal end;
a plunger-holder assembly located within said housing, said plunger-holder assembly configured to receive a plunger of said syringe;
a motor and a screw;
a micro-processor;
a battery;
a solenoid located at the distal end of said housing;
a bearing located above said plunger-holder assembly;
a spring located above said bearing;
a return carriage;
a return carriage spring;
a red sensor;
an LED; and
wherein said red sensor is configured to send a signal to said micro when blood is drawn into the syringe.
